(19) United States
(12) Patent Application Publication
(10) Pub. No.: US 2011/0319771 A1
(43) Pub. Date: Dec. 29, 2011

(54) VITAL LUMINAL PART EVALUATING APPARATUS

(75) Inventors: Hiromasa Tsukahara, Nagoya-shi (JP); Yoshihito Katoh, Nagoya-shi (JP); Takeo Matsumoto, Nagoya-shi (JP); Hiroshi Masuda, Nagoya-shi (JP)

(73) Assignees: UNEX CORPORATION, NAGOYA-SHI, AICHI (JP); NATIONAL UNIVERSITY CORPORATION NAGOYA INSTITUTE OF TECHNOLOGY, NAGOYA-SHI (JP)

(21) Appl. No.: 13/255,353

(22) PCT Filed: Mar. 4, 2010
(86) PCT No.: PCT/JP2010/053585

§ 371 Date: Sep. 8, 2011

(30) Foreign Application Priority Data
Mar. 10, 2009 (JP) 2009-057133

(51) Int. Cl. A61B 5/021

(52) U.S. Cl. 600/485

(57) ABSTRACT

A pressure vessel is provided with an annular inflation bag and an annular inflation bag for sealing the pressure vessel at intermediate positions of brachium and antebrachium of the live body in longitudinal direction of the arms of the live body, and is configured to permit a change of an internal pressure therein over a pressure range a lower limit of which is a negative value, while a portion of the brachium and antebrachium between first and second positions in the longitudinal direction is accommodated in the pressure vessel, so that the pressure vessel can be comparatively small-sized even where arterial vessel (luminal part) of a comparatively large diameter is accommodated in the pressure vessel, whereby the physical and mental burden on the subject person can be reduced.

### TECHNICAL FIELD

The present invention relates to an vital luminal part evaluating apparatus for evaluating a luminal part of a live body, and more particularly to a pressure vessel which accommodates a part of the live body, to change a cross sectional shape of the luminal part.

### BACKGROUND ART

It is well known that evaluation of arterial and venous vessels and other vital luminal parts by objectively measuring dimensions and flexibility of the vital luminal parts by a non-invasion measuring method is effective as information for evaluating a degree of progress of arteriosclerosis, for example, from time to time, and taking a medical treatment before the arteriosclerosis develops into a serious disease such as myocardial infarction, vascular cerebral infarction, obstructive arteriosclerosis and aneurysm.

Known methods for evaluating elasticity of a blood vessel walls include a method wherein a propagation velocity PWV(=L/DT) of a pulse wave is measured on the basis of a time difference DT between two positions on an arterial vessel (artery) spaced apart from each other by a predetermined distance L, to evaluate the arterial vessel in terms of the arteriosclerosis, using the measured propagation velocity PWV, and a method wherein a diameter Ds of the blood vessel at the time of the systolic blood pressure (maximum blood pressure) Ps and a diameter Dd of the blood vessel at the time of the diastolic blood pressure (minimum blood pressure Pd are recorded for each heart beat, and a stiffness parameter β[=In(Ps/Pd)÷(Ds/Dd−1)] is calculated, to evaluate the arterial vessel in terms of the arteriosclerosis, using the calculated stiffness parameter β. Examples of those methods are described in non-patent documents 1 and 2.

For measuring elastic characteristics of the blood vessel wall over a wider range of pressure, there is proposed a method wherein a difference between a depression pressure with which a subject portion of a live body is depressed by a water-inflated bag and a blood pressure of the subject portion is measured as a pressure acting on the blood vessel wall (trans-wall pressure), and the elastic characteristics of the blood vessel wall are measured on the basis of a change of the diameter of the blood vessel when the above indicated blood vessel wall pressure. An example of this method is described in non-patent document 3. According to this method, a physiological pressure range at the time of the measurement, or a range of a difference between the inner and outer pressures of the blood vessel wall, that is, a range of the trans-wall pressure P_{A}(=inner arterial vessel pressure−outer arterial vessel pressure) due to increase of pressure against the blood vessel will is enlarged from a pressure range between a lower limit equal to the diastolic blood pressure and an upper limit equal to the systolic blood pressure, to a pressure range the lower limit of which is lower than the diastolic blood pressure, so that the elastic characteristics of the blood vessel can be measured over the enlarged pressure range.

However, the conventional technology to measure the elastic characteristics of the blood vessel as described above has a drawback that the upper limit of the range of the trans-wall pressure P_{A} in which the elastic characteristics of the blood vessel can be measured is limited to the diastolic blood pressure. Generally, the elastic characteristics of the blood vessel are non-linear, an amount of change of a diameter D of the blood vessel with a change of the blood pressure is abruptly reduced as the blood pressure, that is, the trans-wall pressure P_{A} is raised. This tendency is prominent where the blood vessel suffers from arteriosclerosis. The above-indicated tendency toward the abrupt reduction of the amount of change of the blood vessel diameter with the change of the blood pressure appears particularly in a range corresponding to high blood pressure where the arteriosclerosis of the blood vessel wall is caused by aging of the blood vessel wall. In this respect, it is desired to measure the elastic characteristics of the blood vessel in a range of the trans-wall pressure P_{A} the upper limit of which is higher than the systolic blood pressure, so that the change of the elasticity of the blood vessel can be accurately detected for diagnosis and preventive therapy. However, the method described in the above-indicated patent document 3 does not permit the detection of the elastic characteristics in the range of the trans-wall pressure higher than the systolic blood pressure, so that the elastic characteristics of the luminal parts cannot be detected with a sufficiently high degree of accuracy, resulting in a drawback that the luminal parts cannot be diagnosed in terms of the arteriosclerosis, with a sufficiently high degree of accuracy.

FIG. 17 indicates relationships between the trans-wall pressure P_{A} and a compliance value CC indicative of flexibility of the arterial vessel, for a normal person NAD, a slight arteriosclerosis patient I, a medium arteriosclerosis patient II and a heavy arteriosclerosis patient III. The compliance value CC of the slight arteriosclerosis patient I first increases and then decreases in a pressure range around 100 mmHg, exceeding that of the normal person NAD locally in this pressure range, and continuously decreases in the higher pressure range. That is, a change of the compliance value CC representative of the slight arteriosclerosis patient I does not appear in the pressure range around 100 mmHg, but first appears in the pressure range higher than 150 mmHg. Thus, the conventional method suffers from the drawback that the diagnosis in terms of the arteriosclerosis cannot be effected with a sufficiently high degree of accuracy.

On the other hand, patent document 1 proposes a vital luminal part evaluating apparatus which has a pressure vessel accommodating a portion of a live body and which is configured to change the pressure within the pressure vessel accommodating the portion of the live body, over a pressure range the lower limit of which is a reduced or negative pressure value. Values indicative of a cross sectional shape of a luminal part in the portion of the live body accommodated within the pressure vessel are measured by a non-invasion method by a cross sectional shape measuring device as the pressure within the pressure vessel is changed, and a display is controlled by display control means, to indicate a change of the pressure within the pressure vessel, and a change of the cross sectional shape of the luminal part which takes place with the change of the pressure within the pressure vessel. In this method wherein the pressure within the pressure vessel accommodating the portion of the live body is changed over the pressure range the lower limit of which is the reduced or negative pressure value, the upper limit of the trans-wall pressure of the luminal part which is conventionally limited to the systolic blood pressure can be raised to a value sufficiently higher than the systolic blood pressure, and the change of the pressure within the pressure vessel, and the change of the cross sectional shape of the above-indicated luminal part which takes place with the change of the pressure within the pressure vessel, namely, dynamic characteristics of the luminal part are indicated on the display, on the basis of the values indicative of the cross sectional shape obtained in the pressure range the upper limit of which is sufficiently high, so that the luminal part can be accurately evaluated on the basis of the dynamic characteristics. That is, the elastic characteristics of the luminal part can be detected in the range of the trans-wall pressure the upper limit of which is higher than the systolic blood pressure, so that the elastic characteristics can be accurately obtained, permitting a diagnosis of the luminal part in terms of arteriosclerosis, for example, with a sufficiently high degree of accuracy.

### PRIOR ART DOCUMENT

### Patent Document

Patent Document 1: JP-2008-212366 A

### Non-Patent Documents

Non-patent Document 1:
   “Clinics of Arterial Pulse Wave”, published on Apr. 10, 2003 by Kabushiki Keisha Medical Review, pages 94-95, etc.
Non-patent Document 2:
   “Medical Technology” published on, Jan. 15, 2006 by Medical and Dental Drug Publishing Kabushiki Kaisha, pages 35-40
Non-patent Document 3:
   “In Vivo Human Brachial Artery Elastic Mechanics”; Alan J. Bank et al: Circulation 1999; vol. 100; 41-47
Non-patent Document 4:
   “Biorheology”; 1984; 21(5): 723-34. Richter H A, Mittermayer C; Volume elasticity, modulus of elasticity and compliance of normal and arteriosclerotic human aorta.

### SUMMARY OF THE INVENTION

### Object Achieved by the Invention

By the way, the pressure vessel used in the conventional vital luminal part evaluating apparatus has only one through-hole, through which a portion of the live body is inserted into the pressure vessel. Accordingly, the pressure vessel is required to be large-sized to permit measurement of a change of the shape of a luminal part of a comparatively large diameter selected for improving the accuracy of the measurement, so that a portion of the live body which is depressed within the pressure vessel is increased, resulting in an increase of a physical and mental burden on the subject, which adversely influences the measured values indicative of the cross sectional shape of an arterial vessel, in particular, which is likely to be mentally influenced, making it difficult to ensure sufficiently stable measurement or sufficiently accurate evaluation of the vital luminal part.

The present invention was made in view of the background art described above. It is therefore an object of the present invention to provide a vital luminal part evaluating apparatus which permits accurate evaluation of a luminal part of a live body, with a burden on the live body as small as possible.

### Means for Achieving the Object

The object indicated above is achieved according to the invention of claim 1, which provides a vital luminal part evaluating apparatus (a) which is provided with a pressure vessel configured to permit a change of an internal pressure within a pressure range a lower limit of which is a negative value therein while the pressure vessel accommodates a portion of a live body, and a luminal cross sectional shape measuring device configured to measure, by a non-invasion method, a cross sectional shape value of a luminal part in the portion of the live body accommodated in said pressure vessel, for evaluating said luminal part located in said portion of the live body, on the basis of the cross sectional shape value of the luminal part, and (b) which is characterized in that the above-described pressure vessel is provided with a first sealing device and a second sealing device for sealing the pressure vessel at respective first and second positions in a longitudinal direction of a limb of the above-described live body, and is configured to permit the change of the internal pressure over the pressure range, while a portion of the limb of the above-described live body between the first and second positions is accommodated in the pressure vessel.

### Advantages of the Invention

In the vital luminal part evaluating apparatus according to the invention of claim 1, the pressure vessel is provide with the first sealing device and the second sealing device for sealing the pressure vessel at the respective first and second positions in the longitudinal direction of the limb of the live body, and is configured to permit the change of the internal pressure over the pressure range the lower limit of which is the negative value while the portion of the limb of the live body between the first and second positions is accommodated in the pressure vessel, so that the pressure vessel can be comparatively small-sized even where the luminal part of a comparatively large diameter is accommodated in the pressure vessel, whereby the physical and mental burden on the subject person can be reduced. The reduction of the physical and metal burden permits stable measurement of a cross sectional shape of the luminal part, and consequently permits accurate evaluation of the vital luminal part. It is particularly noted that since the subject person can see the distal part of the limb passed through the pressure vessel, the subject person can be given a high degree of metal stability.

Preferably, the vital luminal part evaluating apparatus is characterized in that the above-described first sealing device and/or the above-described second sealing device are/is provided with an annular inflation bag, which is inflated for sealing at the first position and/or the second position of the limb of the above-described live body, irrespective of whether the pressure vessel accommodates a portion of the limb of the live body between the first and second positions in the longitudinal direction, or an entire distal portion of the limb of the live body. Accordingly, the pressure vessel can be sealed with respect to the external space with high stability, by inflation of the annular inflation bag, irrespective of a dimensional variation of the subject portion of the live body due to sexual, age and physical differences of the live body.

It is also preferable that the vital luminal part evaluating apparatus is characterized in that the above-described first sealing device and/or the above-described second sealing device are/is provided with a pair of flexible annular films which are disposed inside and outside of the above-described pressure vessel and which have radially inner end portions having width dimensions sufficient for surface contact with the above-described limb, for sealing the pressure vessel with respect to the limb of the above-described live body at the first position and/or the second position of the limb, based on a pressure difference between the pressure within the above-described pressure vessel and an atmospheric pressure. Accordingly, the pressure vessel can be sealed with respect to the external space with high stability, at the first position and/or the second position of the limb of the above-described live body, based on the pressure difference between the pressure within the pressure vessel and the atmospheric pressure, irrespective of a dimensional variation of the subject portion of the live body due to sexual, age and physical differences of the live body.

It is also preferable that the above-described vital luminal part evaluating apparatus is characterized by the provision of a display, and display control means for commanding the display to display a change of the internal pressure in the above-described pressure vessel, and a change of a cross sectional shape of the above-described luminal part which takes place with the change of the internal pressure in the pressure vessel. In this case, the cross sectional shape value of the luminal part in a portion of the live body accommodated in the pressure vessel is measured by a non-invasion method by a cross sectional shape measuring device in the process of a change of the internal pressure in the pressure vessel over the pressure range the lower limit of which is a negative value, while the portion of the live body is accommodated in the pressure vessel, and a change of the internal pressure in the pressure vessel and a change of the cross sectional shape of the above-described luminal part which takes place with the change of the interval pressure in the pressure vessel are displayed on the display under the control of the display control means. Since the pressure in the pressure vessel accommodating the portion of the live body is changed over the pressure range the lower limit of which is the negative value, the upper limit of the trans-wall pressure of the luminal part which is conventionally limited to the value corresponding to the systolic blood pressure is raised to a value sufficiently higher than the systolic blood pressure, so that the cross sectional shape value of the luminal part obtained in a high-pressure region of the trans-wall pressure can be used to display on the display the change of the internal pressure in the pressure vessel, and the change of the cross sectional shape of the luminal part with the change of the internal pressure in the pressure vessel, namely, the dynamic characteristics of the luminal part, and to accurately evaluate the part on the basis of the dynamic characteristics. That is, the elastic characteristics of the luminal part can be detected in the high-pressure region of the trans-wall pressure not lower than the systolic blood pressure, so that the elastic characteristics can be accurately obtained, permitting a sufficiently high degree of accuracy of diagnosis in terms of the arteriosclerosis. The upper limit of the trans-wail pressure of the luminal part which is raised to provide the high-pressure region makes it possible to implement the measurement and evaluation while the diameter of the luminal part is enlarged, leading to a further improvement of the measurement accuracy and evaluation accuracy.

It is further preferable that the above-described display control means commands the above-described display to continuously display a plurality of points indicative of a change of the internal pressure in the above-described pressure vessel and a change of the cross sectional shape of the above-described luminal part with the change of the internal pressure in the pressure vessel, in a multi-dimensional coordinate system in which at least the above-described cross sectional shape value and the pressure value in the above-described pressure vessel are indicated as variables. Accordingly, the dynamic characteristics of the luminal part can be obtained on the basis of the points displayed on the display, and the luminal part can be accurately evaluated on the basis of the obtained dynamic characteristics.

It is also preferable that the above-described display control means commands the display to display the internal pressure in the above-described pressure vessel and the cross sectional shape of the above-described luminal part continuously along the axis of time, making it possible to obtain the internal pressure in the pressure vessel and the cross sectional shape value of the above-described luminal part during the measurement, for easy determination of an abnormality of the measurement or rapid treatment of the abnormality.

It is further preferable that the vital luminal part evaluating apparatus includes the pressure control means configured to change the internal pressure in the above-described pressure vessel, between a predetermined negative minimum pressure value and a positive maximum pressure value predetermined to be not lower than the systolic blood pressure of the above-described live body, so that the high-pressure region of the range of the trans-wall pressure can be set as desired by changing the minimum pressure value, to measure the dynamic characteristics of the luminal part in the high-pressure region.

It is also preferable that the above-described cross-sectional-shape measuring device measures at least one of the diameter, wall thickness, perimeter and cross sectional area of the above-described luminal part, on the basis of the reflected ultrasonic wave signal received from the above-described portion of the live body, so that the dynamic characteristics of the luminal part can be accurately obtained on the basis of the measured value or values.

It is further preferable that the vital luminal part evaluating apparatus according to the present embodiment is further arranged such that the cross sectional shape value of the luminal part in the portion of the live body accommodated in the pressure vessel is measured by the non-invasion method by the cross sectional shape measuring device in the process of a change of the internal pressure in the pressure vessel over the pressure range the lower limit of which is a negative value, while the portion of the live body is accommodated in the pressure vessel, and the evaluation values indicative of the dynamic characteristics of the above-described luminal part are calculated by an evaluation value calculating means on the basis of a change of the cross sectional shape of the above-described luminal part which takes place with a change of the internal pressure in the pressure vessel, so that the evaluation values indicative of the dynamic characteristics of the above-described luminal part calculated by the evaluation value calculating means are outputted under the control of output means. Since the pressure in the pressure vessel accommodating the portion of the live body is thus changed over the pressure range the lower limit of which is the negative value, the upper limit of the trans-wall pressure of the luminal part which is conventionally limited to the value corresponding to the systolic blood pressure is raised to a value sufficiently higher than the systolic blood pressure, so that the cross sectional shape value of the luminal part obtained in a high-pressure region of the trans-wall, pressure can be used to calculate the evaluation values indicative of the dynamic characteristics of the luminal part on the basis of the change of the internal pressure in the pressure vessel, and the change of the cross sectional shape of the above-described luminal part with the change of the internal pressure in the pressure vessel, whereby the luminal part can be accurately evaluated on the basis of the dynamic characteristics. That is, the elastic characteristics of the luminal part can be detected in the high-pressure region of the trans-wall pressure not lower than the systolic blood pressure, so that the elastic characteristics can be accurately obtained, permitting a sufficiently high degree of accuracy of diagnosis in terms of the arteriosclerosis. The upper limit of the trans-wall pressure of the luminal part which is raised to provide the high-pressure region makes it possible to implement the measurement and evaluation while the diameter of the luminal part is enlarged, leading to a further improvement of the measurement accuracy and evaluation accuracy.

It is also preferable that the above-described evaluation value calculating means calculates, as an evaluation value or values indicative of the dynamic characteristics of the above-described luminal part, an evaluation value indicative of flexibility of the above-described luminal part and/or an evaluation value indicative of an ability of shrinkage of the above-described luminal body, on the basis of a change of the cross sectional shape of the above-described luminal part which takes place with a change of the internal pressure in the above-described pressure vessel, so that the dynamic characteristics and functions of the luminal part can be accurately obtained on the basis of the calculated evaluation value indicative of the flexibility of the luminal part and/or the calculated evaluation value indicative of the ability of shrinkage of the luminal part.

Preferably, the evaluation values indicative of the flexibility of the above-described luminal part include at least one of a stiffness parameter β, a press-strain elasticity coefficient Ep, an arterial-vessel-diameter change rate AS, a compliance value DC, a compliance value CC and an incremental elasticity coefficient E_{inc}, while the evaluations values indicative of the ability of shrinkage of the above-described luminal part include at least one of a blood vessel shrinkage ratio SR and a time constant τ upon shrinkage of the blood vessel, so that the dynamic characteristics or functions of the luminal part can be accurately obtained.

It is also preferable that the above-described evaluation value calculating means calculates, as evaluation values indicative of the dynamic characteristics of the above-described luminal part, ratios of the evaluation values indicative of the dynamic characteristics of the above-described luminal part obtained in the predetermined high-pressure region of the trans-wall pressure, with respect to those obtained in a predetermined low-pressure region of the trans-wall pressure, so that the luminal part can be accurately evaluated in terms of arteriosclerosis on the basis of the calculated ratios.

It is further preferable that the above-described evaluation value calculating means calculates, as evaluation values indicative of the dynamic characteristics of the above-described luminal part, a ratio of an amount of increase of the cross sectional shape value of the above-described luminal part when the pressure in the above-described pressure vessel is reduced by a predetermined amount, with respect to an amount of decrease of the cross sectionals shape value of the above-described luminal part when the pressure in the above-described pressure vessel is raised by a predetermined amount, so that the luminal part can be accurately evaluated in terms of arteriosclerosis o the basis of the calculated ratio.

Preferably, the luminal part located in a portion of the above-described live body is an arterial vessel in the portion of the live body. In this case, the arterial vessel can be accurately evaluated in terms of arteriosclerosis.

Preferably, the above-described display control means command the display to display graphs indicative of the change of the internal pressure in the pressure vessel and the change of the cross sectional shape of the above-described luminal part which takes place with the change of the internal pressure in the pressure vessel. However, the display control means may be configured to display numerical values indicative of the change of the internal pressure in the pressure vessel and the change of the cross sectional shape of the above-described luminal part which takes place with the change of the internal pressure in the pressure vessel However, the display control means may command the display to display, for instance, a numerical value indicative of a ratio of the change of the internal pressure in the pressure vessel to the change of the cross sectional shape of the luminal part which takes place with the change of the internal pressure in the pressure vessel, or a numerical value indicative of an amount of change of the internal pressure in the pressure vessel, and a numerical value indicative of an amount of change of the cross sectional shape of the luminal part, in comparison with each other.

It is also preferable that the above-described display control means commands the display to continuously display a plurality of points indicative of the change of the internal pressure in the above-described pressure vessel and the change of the cross sectional shape of the above-described luminal part which takes place with the change of the internal pressure in the pressure vessel, in a two-dimensional coordinate system in which a value for the cross sectional shape of the luminal part is taken along an axis while the internal pressure in the above-described pressure vessel is taken along another axis. However, the two-dimensional coordinate system may be replaced by other coordinate systems, such as a polar coordinate system in which the cross sectional shape value and the pressure value in the pressure vessel are indicated by a diameter and an angle. In the coordinate system, the measured values may be represented by a plurality of points lying on curved lines, or a plurality of mutually discrete points.

The maximum and minimum values of the pressure in the pressure vessel which are used by the pressure control means to control the pressure in the above-described pressure vessel and which respectively correspond to the lower and upper limits of the pressure range in which the trans-wall pressure is changed, and the blood pressure values of the live body used to calculate the stiffness parameter may be measured before the pressure control and manually entered for use by the pressure control means. Preferably, blood pressure measuring means is provided to automatically measure the blood pressure values of the live body, on the basis of the pulse wave generated by the arterial vessel in a portion of the live body when the pressure of depression of that portion of the live body is changed, or on the basis of a change of an an of the shape of the arterial vessel, so that the maximum value and/or the minimum value of the pressure in the pressure vessel is/are automatically calculated on the basis of the measured blood pressure values. The maximum value of the pressure in the pressure vessel is determined to be equal to the systolic blood pressure of the live body, for example. The minimum value (negative value) of the pressure in the pressure vessel is determined to be equal to the predetermined upper limit of the trans-wail pressure of about 200-250 mmHg minus the systolic blood pressure. This systolic blood pressure (maximum blood pressure) may be replaced by the diastolic blood pressure.

Preferably, the cross sectional shape value of the above-described luminal part is a diameter or a wall thickness of the luminal part. However, the cross sectional shape value may be a perimeter or cross sectional area of the luminal part. In essence, the cross sectional shape value should relate to a size of the cross sectional shape of the luminal part.

Preferably, a portion of the above-described live body is depressed by a cuff when the blood pressure is measured by the above-described blood pressure measuring means. However, the portion of the live body may be depressed by using the above-described pressure vessel. In this case wherein the pressure vessel is also used for depression of the portion of the live body, the cuff and a pressure control valve for controlling the pressure in the cuff may be eliminated.

While the above-described luminal part of the live body is preferably an arterial vessel located in the above-described portion of the live body, the luminal part may be a circulatory organ such as a venous vessel, a respiratory organ such as a lung, a digestive organ, or an urinary bladder. The limb of the live body may be a wrist, a brachium, a leg, a thigh or a foot, as well as an antebrachium.

### BRIEF DESCRIPTION OF THE DRAWINGS

FIG. 1 is a block diagram schematically showing an arrangement of a vital luminal part evaluating apparatus according to one embodiment of this invention;

FIG. 2 is a longitudinal cross sectional view showing an arrangement of a pressure vessel shown in FIG. 1;

FIG. 3 is a transverse cross sectional view taken in a direction of arrows of FIG. 2, showing the arrangement of the pressure vessel shown in FIG. 1;

FIG. 4 is a right-side view showing of the arrangement of the pressure vessel shown in FIG. 1;

FIG. 5 is a view showing examples of display of a diameter and a wall thickness of an arterial vessel from time to time during measurement, under the control of a display control portion shown in FIG. 1;

FIG. 6 is a view showing an example of a graph indicating a relationship between the diameter and a trans-wall pressure of the arterial vessel, namely, dynamic characteristics of the arterial vessel, which are displayed after the measurement, under the control of the display control portion shown in FIG. 1;

FIG. 7 is a view showing an example of a graph indicating a relationship between the thickness of the arterial vessel and a trans-wall pressure, namely, dynamic characteristics of the arterial vessel, which are displayed after the measurement, under the control of the display control portion shown in FIG. 1;

FIG. 8 is a view showing an example of a graph indicating a relationship between the diameter of the arterial vessel and a pressure of the pressure vessel, namely, dynamic characteristics of the arterial vessel, which are displayed after the measurement, under the control of the display control portion shown in FIG. 1;

FIG. 9 is a view showing an example of a graph indicating a relationship between the diameter of the arterial vessel and a pressure of the pressure vessel, namely, dynamic characteristics of the arterial vessel, which are displayed after the measurement, under the control of the display control portion shown in FIG. 1;

FIG. 10 is a flow chart illustrating a major portion of an operation to control a main body of the vital luminal part evaluating apparatus of FIG. 1;

FIG. 11 is a view indicating a change of a diameter D of the arterial vessel with a change of a depression pressure acting on the arterial vessel during measurement of elastic characteristics of the blood vessel wall on the basis of the change of the blood vessel diameter with a change of a pressure acting on the blood vessel wall (trans-wall pressure), which is a difference between the blood pressure and the depression pressure with which the subject portion of the live body is depressed by water-inflated bags;

FIG. 12 is a view indicating a so-called “Bayliss effect” wherein the diameter of the arterial vessel of the subject within the evacuated pressure vessel once increases and then decreases along a logarithmic curve;

FIG. 13 is a longitudinal cross sectional view showing an arrangement of sealing devices provided in a pressure vessel according to another embodiment of this invention;

FIG. 14 is a longitudinal cross sectional view showing an arrangement of sealing devices provided in a pressure vessel according to a further embodiment of the invention;

FIG. 15 is a time chart for explaining an operation of a blood pressure measuring portion to effect blood pressure measurement using a pressure vessel;

FIG. 16 is a time chart for explaining an alternative operation of the blood pressure measuring portion to effect the blood pressure measurement using the pressure vessel; and

FIG. 17 is a view indicating relationships between a compliance value and a trans-wall pressure of an arterial vessel, for a normal person, a slight arteriosclerosis patient, a medium arteriosclerosis patient and a heavy arteriosclerosis patient.

### BEST MODE FOR CARRYING OUT THE INVENTION

An embodiment of a vital luminal part evaluating apparatus 10 of the present invention will be described by reference to the drawings.

### Embodiment 1

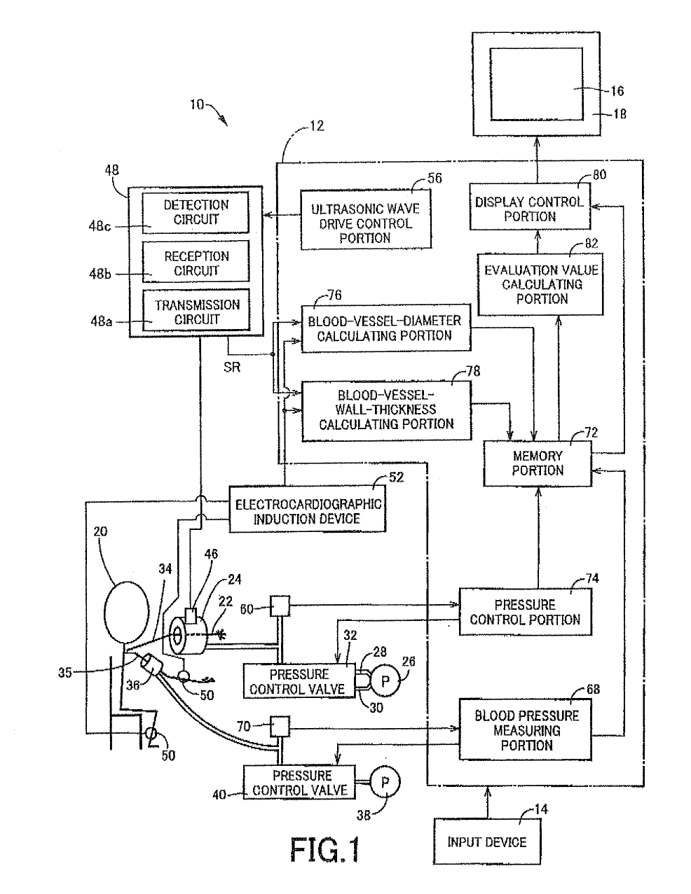

FIG. 1 is a block diagram showing an arrangement of a vital luminal part evaluating apparatus 10. The vital luminal part evaluating apparatus 10 is provided with a main body (electronic control device) 12, an input device 14 having a keyboard, mouse, etc. for inputting operation signals into the main body 12, and an image display device 18 having a display 16 capable of displaying images such as graphic images and symbols on the basis of output signals of the main body 12. The main body 12 is constituted by a so-called “microcomputer” which incorporates a CPU, a ROM, a RAM and an input/output interface and which processes input signals under the control of the CPU according to control programs stored in the ROM while utilizing a temporary data storage function of the RAM. Control functions of the main body 12 described above are indicated by a plurality of functional blocks.

The vital luminal part evaluating apparatus 10 is also provided with a pressure vessel 24, and pressure control valves 32 and 40. The pressure vessel 24 is configured to accommodate a brachium or forearm 34 of a subject person (live body) 20. The pressure control valve 32 connects the pressure vessel 24 selectively to one of a suction conduit 28 and a delivery conduit 30 of a pneumatic pump 26, for controlling a pressure in the pressure vessel 24 within a pressure range between a reduced or negative pressure value and an elevated or positive pressure value. The pressure control valve 40 controls a pressure in a cuff 36 wound on another brachium 35 of the subject person (live body) 20 for measuring a blood pressure of the subject person 20, by controlling an output pressure of a pneumatic pump 38.

The vital luminal part evaluating apparatus 10 is further provided with an ultrasonic wave probe (ultrasonic wave contact member) 46, an ultrasonic wave drive control device 48 and an electrocardiographic induction device 52. The ultrasonic wave probe 46 is supported by the pressure vessel 24 such that the ultrasonic wave probe 46 contacts a skin 42 of the brachium 34, to detect a cross sectional image (cross sectional shape) of an arterial vessel 44 right under the skin 42. The ultrasonic wave drive control device 48 is configured to command the ultrasonic wave probe 46 to generate an ultrasonic wave and receive a reflected wave, and to apply a reflected ultrasonic wave signal SR to the main body 12. The electrocardiographic induction device 52 is provided with a plurality of electrodes 50 to be held on the subject person 20, and is configured to receive an electrocardiographic induction signal generated in synchronization with a heart beat of the subject person 20, and to apply the generated electrocardiographic induction signal to the main body 12. The ultrasonic wave probe 46 described above is provided on its lower or pressing surface with a multiplicity of oscillators (e.g., piezoelectric ceramic chips), which are usually arranged in an array along a straight line intersecting a direction of extension of the arterial vessel 44. The ultrasonic wave drive control device 48 described above is provided with a transmission circuit 48a, a reception circuit 48b and a detection circuit 48c. The transmission circuit 48a is configured to sequentially drive different groups of the oscillators, for irradiating the ultrasonic wave, and the reception circuit 48b is configured to command the oscillators to receive the wave reflected from a tissue of the live body, and to receive the reflected wave from the oscillators, while the detection circuit 48c is configured to detect a signal received from the reception circuit 48b and to apply the detected signal to the main body 12. The above-described ultrasonic wave probe 46 constitutes a part of cross-sectional-shape measuring device.

An ultrasonic wave drive control portion 56 of the main body 12, which corresponds to ultrasonic wave drive control means, is configured to operate according to a predetermined control program, for implementing a beam forming drive of the multiple ultrasonic wave oscillators (piezoelectric chips) arranged in a line to form the ultrasonic array upon each reception of the electrocardiographic induction signal from the electrocardiographic induction device 52 synchronizedly, such that each group of the ultrasonic wave oscillators irradiates a convergent ultrasonic wave beam toward the arterial vessel 44 at a frequency of about 10 MHz sequentially in the direction of arrangement of the ultrasonic wave oscillator, while giving a predetermined phase difference for each of the different groups of the ultrasonic wave oscillators, from one end of the array, each group consisting of a predetermined number of the oscillators, so that the signal corresponding to the wave reflected upon each irradiation of the ultrasonic wave beam is received by the main body 12. The array of the ultrasonic wave oscillators is provided on its irradiation surface with an acoustic lens for converging the ultrasonic wave beam in a direction perpendicular to the direction of arrangement of the ultrasonic wave oscillators.

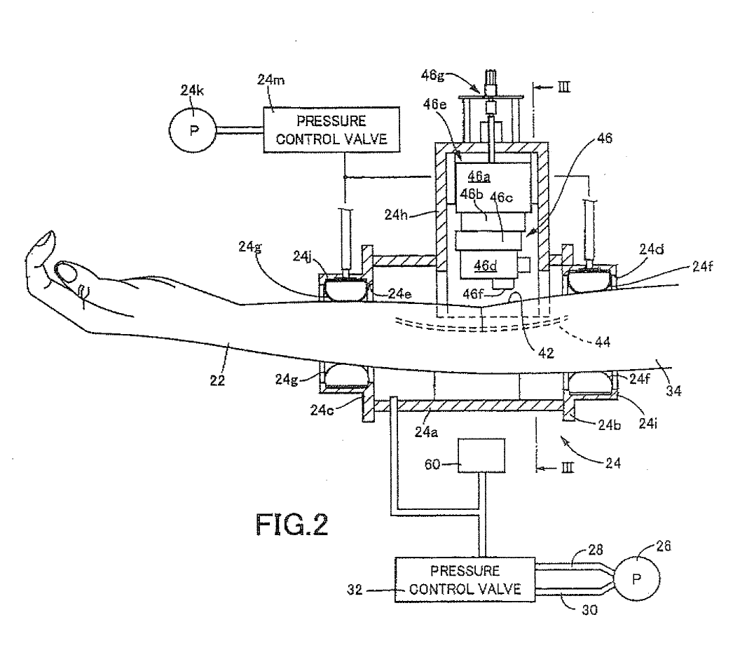

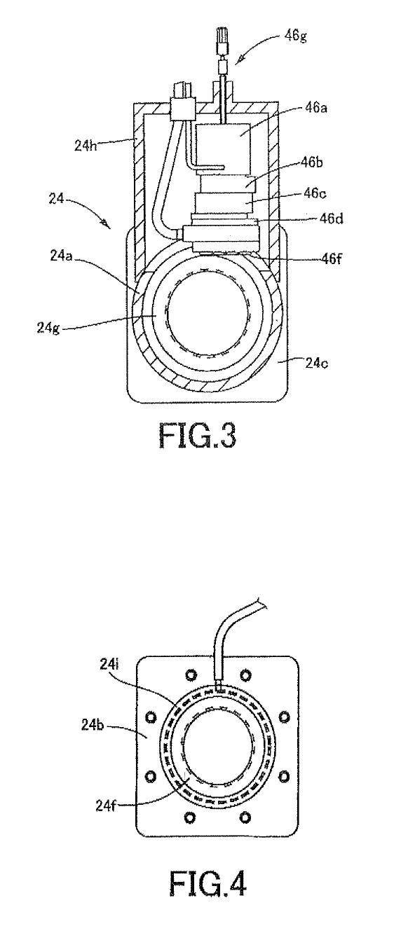

FIG. 2 is the longitudinal cross sectional view of the pressure vessel 24, and FIG. 3 is the transverse cross sectional view of the pressure vessel 24, while FIG. 4 is the side view (end view) of the pressure vessel 24. The pressure vessel 24 is comparatively air-tightly formed by a cylindrical outer wall 24a constituted by a tubular member, and a pair of end walls 24b, 24c air-tightly closing the respective opposite open ends of the outer wall 24a. The pair of end walls 24b, 24c have a pair of cylindrical portions 24i, 24j protruding outwardly, a pair of through-holes 24d, 24e formed on a pair of end walls 24b, 24c and continuously with inner circumferential surfaces of the cylindrical portions 24i, 24j, to permit one of the four limbs of the live body, for example, an antebrachium or forearm 22 to pass through, and a pair of annular inflation bags 24f, 24g formed of a soft resin or synthetic rubber material and fixed to the inner circumferential surfaces of the through-holes 24d, 24e, for sealing between the through-hole 24d and the brachium 34 and between the through-hole 24e and the antebrachium 22. These annular inflation bags 24f, 24g respectively function as a first sealing device and a second sealing device for sealing between the pressure vessel 24 and the relevant limb in the form of the brachium or antebrachium at respective first and second positions that are spaced apart in the longitudinal direction of the limb.

The pressure vessel 24 is provided at upper portion of the cylindrical outer wall 24 with a box structure wall 24h in the form of a rectangular box which extends upwards and in which the ultrasonic wave probe 46 is accommodated such that the ultrasonic wave probe 46 contacts the skin 42 of the brachium 34 within the pressure vessel 24. This ultrasonic wave probe 46 has an ultrasonic wave array contact member 46f installed within the pressure vessel 24 through a multiple-axes drive device 46e. The multiple-axes drive device 46e consists of a base 46a fixed to the pressure vessel 24 through a vertical position adjusting mechanism 46g, an oscillation angle adjusting device 46b for adjusting an angle of oscillation about an oscillation axis which is parallel to an X-axis direction perpendicular to the direction of extension of the arterial vessel 44 and which passes adjacent to the arterial vessel 44, an X-axis position adjusting device 46c for adjusting the position in the X-axis direction, and a rotation angle adjusting device 46d for adjusting an angle of rotation about a vertical axis. For example, the ultrasonic wave array contact member 46f consists of three ultrasonic wave arrays arranged in the form of a letter H, that is, a pair of short ultrasonic wave arrays parallel to each other, and one long ultrasonic wave array interposed between the two short ultrasonic wave arrays, and is fixed to a lower surface of the rotation angle adjusting device 46d, namely, to a contact surface for contact with the skin 42 of the brachium 34.

The annular inflation bags 24f, 24g fixed to the inner circumferential surfaces of the through-holes 24d, 24e of the above-described pressure vessel 24 are connected to a pressure control valve 24m, which controls an output pressure of a pneumatic pump 24k to control pressures in the annular inflation bags 24f, 24g. As a result of inflation of the above-described pair of annular inflation bags 24f, 24g, the inside diameters of the annular inflation bags 24f, 24g are reduced so that air tightness is established between the through-hole 24d and the brachium 34 and between the through-hole 24e and the antebrachium 22, whereby the pressure vessel 24 is air-tightly sealed. In response to insertion of the Brachial into the pressure vessel 24 before a measurement operation, which initiates the measurement operation, the electronic control device 12 controls the pressure control valve 24m to inflate the above-described pair of annular inflation bags 24f, 24g, for thereby establishing air tightness between the pressure vessel 24 and the brachia 34, 22.

Referring back to FIG. 1, a blood pressure measuring portion 68 of the main body 12, which corresponds to blood pressure measuring means, is configured to perform an operation to measure a blood pressure of the subject person 20 by an oscillometric method using the cuff 36, before measurement of dynamic characteristics of the arterial vessel and evaluation of a degree of arteriosclerosis of the arterial vessel. Namely, the blood pressure measuring portion 68 controls the pressure control valve 40 to control the pressure of the cuff 36 as detected by a pressure sensor 70, such that the pressure of the cuff 36 is initially raised to a hemostatic pressure higher than the systolic blood pressure (maximum blood pressure) of the subject person 20, and is then gradually reduced at a predetermined rate. In this process of change of the pressure of the cuff 36, the blood pressure measuring portion 68 extracts a pressure pulsation wave, that is, a pulse wave generated in synchronization with the heart beat, and determines, as a systolic blood pressure Ps and a diastolic blood pressure Pd, the pressure values of the cuff 36 corresponding to inflection points of an envelope connecting amplitude values of the pulse wave, namely, the pressure values of the cuff 36 corresponding to maximum values of a difference of the amplitude values of the pulse wave. The blood pressure measuring portion 68 stores the determined systolic and diastolic blood pressures Ps and Pd in a memory portion 72.

A pressure control portion 74 of the main body 12, which corresponds to pressure control means, is configured to operate upon measurement of the dynamic characteristics of the arterial vessel and evaluation of the degree of arteriosclerosis of the arterial vessel, to change a pressure Pc within the pressure vessel 24 over a predetermined range the lower limit of which is a reduced or negative pressure value, that is, to change a difference between the pressures acting on the inner and outer sides of the arterial vessel 44, namely, to change a trans-wall pressure P_{A} (inner side pressure of the arterial vessel−outer side pressure of the arterial vessel), from a predetermined lower limit which is a reduced or negative pressure value, to a predetermined upper limit of about 200-250 mmHg, such that the trans-wall pressure P_{A} is repeatedly changed in the opposite directions between the lower and upper limits. It is reasonable to measure a change of the cross sectional shape of the arterial vessel 44 over a range of the trans-wall pressure P_{A} between the lower limit at which the arterial vessel 44 has a minimum cross sectional area, and the upper limit at which the arterial vessel 44 has a maximum cross sectional area. In view of this, the pressure control portion 74 gradually changes the trans-wall pressure P_{A} from the lower limit of 0 mmHg at which the pressure Pc in the pressure vessel 24 has a maximum value equal to the diastolic blood pressure Pd when the inner side pressure of the arterial vessel 44 is equal to the diastolic blood pressure Pd, to the upper limit of about 200-250 mmHg at which the pressure Fe in the pressure vessel 24 has a minimum value that is a negative value of about −80 mmHg, for example, when the inner side pressure of the arterial vessel 44 is equal to the systolic blood pressure Ps. The minimum pressure value (negative pressure value) in the pressure vessel 24 is determined to be a difference obtained by subtracting the predetermined upper limit of the trans-wall pressure P_{A} from the systolic blood pressure Ps. The above-indicated diastolic blood pressure Pd and systolic blood pressure Ps are those measured by the blood pressure measuring portion 68 and stored in the memory portion 72. Where the blood pressure measuring portion 68 is not provided, the diastolic and systolic blood pressures measured for this purpose are manually entered.

A blood-vessel-diameter calculating portion 76 of the main body 12, which corresponds to blood-vessel-diameter calculating means, is configured to receive the reflected ultrasonic wave signal SR through a gate which is opened each time the electrocardiographic induction signal is received from the electrocardiographic induction device 52, and to process the received reflected ultrasonic wave signal SR in synchronization with the electrocardiographic induction signal, for repeatedly calculating a diameter D (mm) of the arterial vessel 44 and storing from time to time, in the memory portion 72, the calculated diameter D together with the pressure Pc in the pressure vessel 24 and the trans-wall pressure P_{A}. The wall of the arterial vessel 44 has a portion relatively near the ultrasonic wave probe 46, and a portion relatively distant from the ultrasonic wave probe 46, in the direction of diameter of the arterial vessel 44, and the above-indicated reflected ultrasonic wave signal SR includes a first reflected wave reflected from the wall portion relatively near the ultrasonic wave probe 46, and a second reflected wave reflected from the wall portion relatively distant from the ultrasonic wave probe 46. For example, the blood-vessel-diameter calculating portion 76 calculates the outside diameter (blood vessel diameter) D of the arterial vessel 44 on the basis of a time lag between the leading end of the first reflected wave and the trailing end of the second reflected wave, and a predetermined velocity of propagation of the ultrasonic wave through the relevant tissue of the live body. On the basis of the reflected ultrasonic wave signal SR, a cross sectional image of the arterial vessel 44 is also generated to obtain the diameter D of the arterial vessel 44 on the basis of the cross sectional image of the arterial vessel 44.

A blood-vessel-wall-thickness calculating portion 78 of the main body 12, which corresponds to blood-vessel-wall-thickness calculating means, is configured to receive the reflected ultrasonic wave signal SR through a gate which is opened each time the electrocardiographic induction signal is received from the electrocardiographic induction device 52, and to process the received reflected ultrasonic wave signal SR in synchronization with the electrocardiographic induction signal, for repeatedly calculating a wall thickness T (mm) of the arterial vessel 44 and storing from time to time, in the memory portion 72, the calculated wall thickness T together with the pressure Pc in the pressure vessel 24 and the trans-wall pressure P_{A}. For example, the blood-vessel-wall-thickness calculating portion 78 calculates the wall thickness T of the arterial vessel 44 on the basis of a time lag between the leading and trailing ends of the above-indicated first reflected wave or a time lag between the leading and trailing ends of the above-indicated second reflected wave, and the predetermined velocity of propagation of the ultrasonic wave through the relevant tissue of the live body. On the basis of an ultrasonic wave image or the time lag of the first and second reflected waves, for example, the outside diameter D and an inside diameter d of the arterial vessel 44 are obtained, and the wall thickness T(=(D−d)/2) of the arterial vessel 44 is calculated on the basis of the difference between the outside and inside diameters D and d. Where the above-described electrocardiographic induction device 52 is not used, the ultrasonic wave is repeatedly irradiated and received more than ten times per second, and the maximum value of the outside diameter D is determined as an outside diameter Ds corresponding to the systolic blood pressure, while the minimum value of the outside diameter D is determined as an outside diameter Dd corresponding to the diastolic blood pressure, and the maximum value of the wall thickness T is determined as a wall thickness Ts corresponding to the diastolic blood pressure, while the minimum value of the wall thickness T is determined as a wall thickness Td corresponding to the systolic blood pressure.

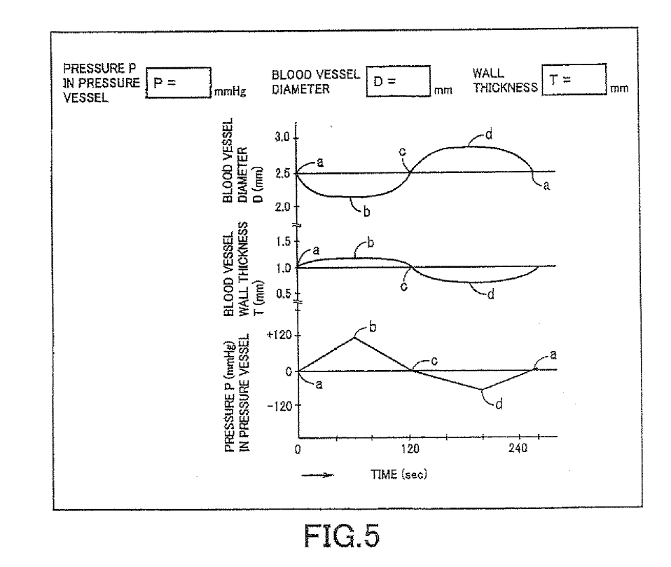

A display control portion 80 of the main body 12, which corresponds to display control means, is configured to command the display 16 to display from time to time values indicative of the pressure Pc in the pressure vessel 24, and the diameter D and wall thickness T of the arterial vessel 44, and a trend graph indicative of changes of those values with the time, as shown in FIG. 5, while the pressure Pc in the pressure vessel 24 is changed under the control of the pressure control portion 74 to measure the diameter D and wall thickness T. The display 16 displays the diameter D and wall thickness T, on the basis of the values D, T stored in the memory portion 72 together with the pressure Pc and trans-wall pressure P_{A}.

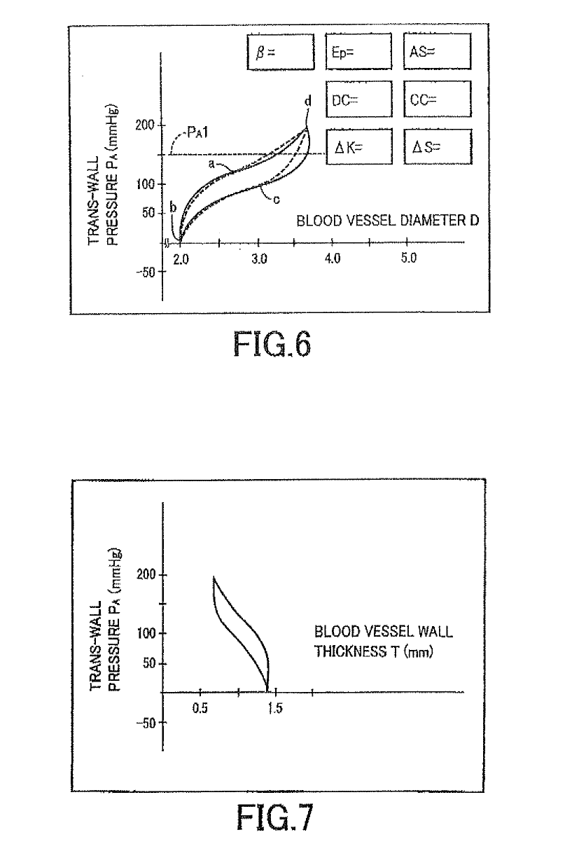

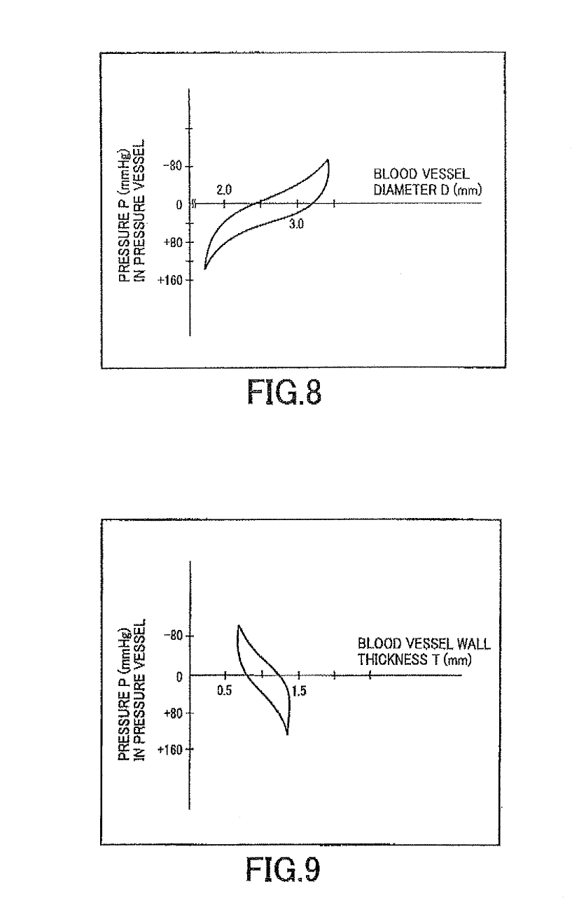

The above-described display control portion 80 commands the display 16 to display a graph of FIG. 6 indicative of a change of the diameter D of the arterial vessel 44 with the trans-wall pressure P_{A}, a graph of FIG. 7 indicative of a change of the wall thickness T of the arterial vessel 44 with the trans-wall pressure P_{A}, a graph of FIG. 8 indicative of a change of the diameter D of the vessel with the pressure Pc in the pressure vessel 24, and a graph of FIG. 9 indicative of a change of the wall thickness T of the vessel with the pressure Pc in the pressure vessel 24, all together, or selectively according to a manual selecting operation, on the basis of the pressure Pc and the diameter D and wall thickness T of the arterial vessel 44 which are measured and stored in the memory portion 72 from time to time, while the pressure Pc in the pressure vessel 24, that is, the difference between the pressures acting on the inner and outer sides of the arterial vessel 44, namely, the trans-wall pressure P_{A} (inner side pressure of the arterial vessel−outer side pressure of the arterial vessel) is changed under the control of the pressure control portion 74, over the predetermined range the lower limit of which is a reduced or negative pressure value, that is, changed, for example, from the predetermined lower limit which is a reduced or negative pressure value, to the predetermined upper limit of about 200-250 mmHg, such that the trans-wall pressure P_{A} is repeatedly changed in the opposite directions between the lower and upper limits. These graphs represent continuous curves obtained by converting data plots by interpolation, but may represent discrete points of the data plots as they are. The graphs indicate the dynamic characteristics of the arterial vessel 44 relating to its flexibility or stiffness, and can be used to evaluate the arterial vessel 44 in terms of the degree of stiffness.

In FIG. 6, for example, broken lines indicate the dynamic characteristics of the arterial vessel of the normal person, while solid lines indicate the dynamic characteristics of arterial vessel of the arteriosclerotic patient. In a high-pressure region of the trans-wall pressure P_{A} of 120-200 mmHg, the trans-wall pressure P_{A} indicated by the solid lines abruptly increases with an increase of the blood vessel diameter D, and this abrupt increase of the trans-wall pressure P_{A} indicates a relatively high degree of stiffness of the arterial vessel 44, while the trans-wall pressure indicated by the broken lines relatively gradually increases with the increase of the blood vessel diameter D, and this gradual increase of the trans-wall pressure P_{A} indicates a relatively high degree of softness of the arterial vessel 44. It is noted that the blood vessel diameter D indicated by the broken and solid lines in FIG. 6 is normalized by a radius of the blood vessel at 0 mmHg.

An evaluation value calculating portion 82 of the main body 12, which corresponds to evaluation value calculating means, is configured to calculate values indicative of the dynamic characteristics of the arterial vessel 44, that is, values to be used for evaluating the arterial vessel 44 in terms of the degree of stiffness, such as a stiffness parameter β, a press-strain elasticity coefficient Ep, an arterial-vessel-diameter change rate AS, a compliance value DC, a compliance value CC, an incremental elasticity coefficient E_{inc}, and a blood vessel shrinkage ratio SR, for example, according to the following Equations (1) through (7), for calculating a time constant τ upon shrinkage of the blood vessel, in the high-pressure region of the trans-wall pressure P_{A} not lower than 120-150 mmHg, for instance, while the pressure Pc in the pressure vessel 24, that is, the difference between the pressures acting on the inner and outer sides of the arterial vessel 44, namely, the trans-wall pressure P_{A} (inner side pressure of the arterial vessel−outer side pressure of the arterial vessel) is changed under the control of the pressure control portion 74, over the predetermined range the lower limit of which is a reduced or negative pressure value, that is, changed from the predetermined lower limit which is a reduced or negative pressure value, to the predetermined upper limit of about 200-250 mmHg, such that the trans-wall pressure P_{A} is repeatedly changed in the opposite directions between the lower and upper limits. In the Equations (1) through (7), Ps′, Pd′, Ds′, Dd′, D, ΔD(=Ds′−Dd′), ΔP(=Ps′−Pd′), and In represent the following values:
Ps′: a trans-wall pressure during the systole
Pd′: a trans-wall pressure during the diastole
Ds′: a blood vessel diameter during the systole
Dd′: a blood vessel diameter during the diastole
D: a diameter selected within a range between Ds′ and Dd′
ΔD: an amount of change of the blood vessel diameter
ΔP: a puke pressure
In: a natural logarithm
   In the Equation (6), D₀, Di and v represent the following values:
D₀: a blood vessel outside diameter
Di: a blood vessel inside diameter
V: a Poisson's ratio
   In the Equation (7), ΔD₂ and ΔD₁ represent the following values:
ΔD₂: an amount of increase of the diameter of the arterial vessel 44 when the pressure Pc in the pressure vessel 24 is reduced to a negative pressure value
ΔD₁: an amount of decrease of the diameter of the arterial vessel 44 upon elapsing of a predetermined time after the pressure Pc is reduced to the negative pressure value.

β=In(Ps′/Pd′)/(ΔD/Dd′) (1)

Ep=ΔP/(ΔD/D) (2)

AS=ΔD/D (3)

DC=(2ΔD/D)/ΔP (4)

CC=πD(ΔD/2ΔP) (5)

E_{inc}=ΔP·2(1−v²)D₀Di²/{ΔD(D₀²−Di²)} (6)

SR=ΔD₂/ΔD₁ (7)

FIG. 12 indicates a phenomenon in which the diameter D of the arterial vessel 44 increases when the pressure Pc in the pressure vessel 24 is reduced to a negative pressure value and subsequently decreases along a logarithmic curve owing to an action of the smooth muscle. This phenomenon is referred to as a “Bayliss effect” or a “Myogenic response”. The above-described blood vessel shrinkage ratio SR represents a shrinkage ability of the smooth muscle relating to a state of health of the blood vessel (arterial vessel stiffness state). For instance, the above-described time constant τ upon shrinkage of the blood vessel is a length of time lapse from a moment at which the pressure Pc in the pressure vessel 24 is reduced to a negative pressure value, as indicated in. FIG. 12, and is obtained by measuring the length of time to a moment at which the blood vessel diameter as represented by a decrease curve has deceased to a value of 0.368×ΔD₂, or by curve-fitting a logarithmic attenuation curve on the decrease curve of diameter of the blood vessel.

The evaluation value calculating portion 82 is also configured to calculate, as values indicative of the dynamic characteristics of the arterial vessel 44, differences or ratios AK of the stiffness parameter β, press-strain elasticity coefficient Ep, arterial-vessel-diameter change rate AS, compliance value DC, compliance value CC, incremental elasticity coefficient E_{inc}, blood vessel shrinkage ratio SR and time constant τ upon shrinkage of the blood vessel in the high-pressure region of the trans-wall pressure P_{A} not lower than 120-150 mmHg, for example, with respect to those in a low-pressure region of the trans-well pressure P_{A} not higher than 80 mmHg.

The evaluation value calculating portion 82 is further configured to calculate, as a value indicative of the dynamic characteristics of the arterial vessel 44, a ratio ΔS of an amount of increase ΔD⁺ of the blood vessel diameter D when the pressure in the pressure vessel 24 is reduced by a predetermined amount in the above-indicated high-pressure region, with respect to an amount of decrease ΔD⁻ of the blood vessel diameter D when the pressure in the pressure vessel 24 is raised by a predetermined amount in the above-indicated high-pressure region

The above-described display control portion 80 commands the display 16 to display, as indicated in FIG. 6, the stiffness parameter β, press-strain elasticity coefficient Ep, arterial-vessel-diameter change rate AS, compliance value DC, compliance value CC, incremental elasticity coefficient E_{inc}, blood vessel shrinkage ratio SR, and time constant τ upon shrinkage of the blood vessel, or the ratios ΔK and/or ratio ΔS, which are calculated by the above-described evaluation value calculating portion 82 by using data obtained at a predetermined value trans-wall pressure value P_{A}1, for example, at 150 mmHg within the high-pressure region, while the pressure Pc in the pressure vessel 24, that is, the difference between the pressures acting on the inner and outer sides of the arterial vessel 44, namely, the trans-wall pressure PA (inner side pressure of the arterial vessel−outer side pressure of the arterial vessel) is changed under the control of the pressure control portion 74, over the predetermined range the lower limit of which is a reduced or negative pressure value, that is, changed, for example, from the predetermined lower limit which is a reduced or negative pressure value, to the predetermined upper limit of about 200-250 mmHg, such that the trans-wall pressure P_{A} is repeatedly changed in the opposite directions between the lower and upper limits.

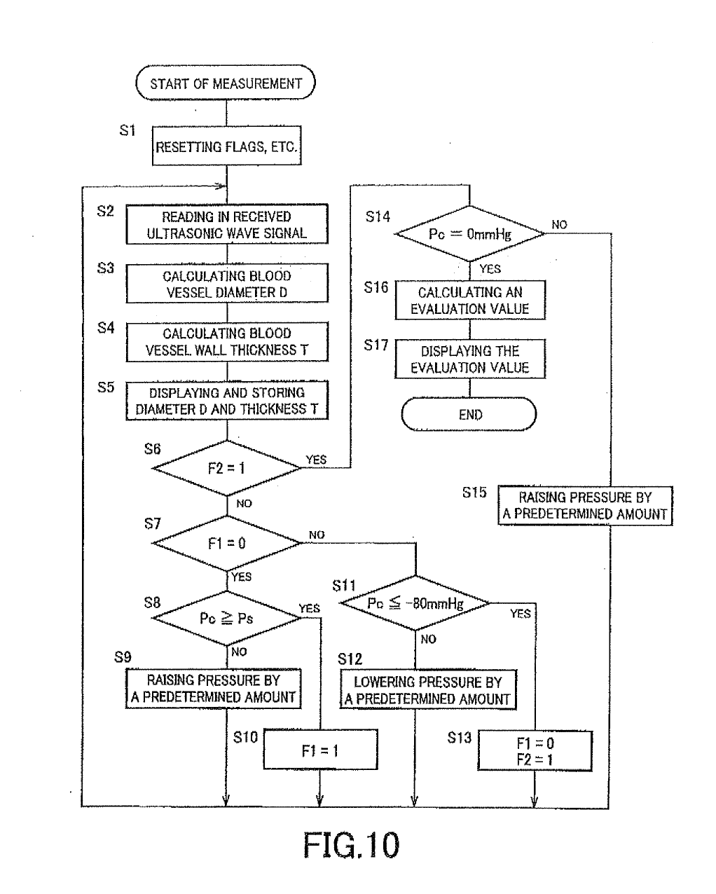

FIG. 10 is the flow chart illustrating a blood vessel dynamic characteristics measurement control operation performed by the main body 12, which functions as the electronic control device. The control operation is initiated when a manual operation to start the control operation is performed while the brachium 34 of the subject person 20 is accommodated in the pressure vessel 24 such that the ultrasonic wave probe 46 is located on the arterial vessel 44 within the brachium 34.

Referring to FIG. 10, step S1 (hereinafter “step” being omitted) is implemented to reset flags, etc., and S2 is then implemented to read in the reflected ultrasonic wave signal SR. Subsequently, S3 corresponding to the above-described blood-vessel-diameter calculating portion 76 is implemented to process the reflected ultrasonic wave signal SR, for calculating the diameter D (mm) of the arterial vessel 44 right under the ultrasonic wave probe 46, and to store the calculated diameter D in the memory portion 72. Then, S4 corresponding to the above-described blood-vessel-wall-thickness calculating portion 78 is implemented to process the reflected ultrasonic wave signal SR, for calculating the wall thickness T (mm) of the arterial vessel 44 right under the ultrasonic wave probe 46, and to store the calculated wall thickness T in the memory portion 72. Subsequently, S5 corresponding to the display control portion 80 is implemented to display numerical values indicative of the calculated diameter D and wall thickness T of the arterial vessel 44, together with the pressure Pc in the pressure vessel 24 at the time of calculation, and corresponding graphs indicative of their chronological changes, as shown in FIG. 5.

S6 is then implemented to determine whether the pressure Pc in the pressure vessel 24 is 0 mmHg (whether the trans-wall pressure P_{A} is equal to the systolic blood pressure Ps) while a re-evacuation progress flag F2 is set at 1. Since a negative determination is obtained in S6 immediately after initiation of the measurement control operation, the control flow goes to S7 to determine whether a re-pressurization progress flag F1 is set at 0. Since an affirmative determination is obtained in S7 immediately after initiation of the measurement control operation, the control flow goes to S8 to determine whether the pressure Pc in the pressure vessel 24 has become equal to or higher than the upper limit equal to the systolic blood pressure Ps (whether the trans-wall pressure P_{A} has been reduced to 0 mmHg or lower). Since a negative determination is obtained in S8 immediately after initiation of the measurement control operation, the control flow goes to S9 corresponding to the above-described pressure control portion 74, to raise the pressure Pc in the pressure vessel 24, by a predetermined incremental amount ΔPc1, for example by about 1-20 mmHg. If the incremental amount ΔPc1 is predetermined to be about 1 mmHg, the pressure Pc is considered to be continuously raised. In the incremental amount ΔPc1 is predetermined to be about 10-20 mmHg, the pressure Pc is considered to be raised in steps. Then, the control cycle starting with the above-described S2 followed by the subsequent steps is repeatedly executed, so that the diameter D and wall thickness T of the arterial vessel 44 are repeatedly calculated while the pressure Pc in the pressure vessel 24 is raised from time to time. This control cycle corresponds to a period from a point of time “a” to a point of time “b” indicated in FIGS. 5 and 6.

When the pressure Pc in the pressure vessel 24 has been raised to the systolic pressure Ps (when the trans-wall pressure Pa has been reduced to 0 mmHg or lower) during repeated execution of the above-described control cycle, an affirmative determination is obtained in S8, and the control flow goes to S10 to set the re-pressurization progress flag F1 to 1. Accordingly, a negative determination is obtained in S7 of the control cycle starting with S2, and the control flow goes to S11 to determine whether the pressure Pc in the pressure vessel 24 has been reduced to the lower limit of −80 mmHg (whether the trans-wall pressure P_{A} has been raised to its maximum value (Ps+80 mmHg), for example, 200 mmHg or higher). Since a negative determination is obtained in S11 when this step is implemented for the first time, the control flow goes to S12 to corresponding to the above-described pressure control portion 74, to reduce the pressure Pc in the pressure vessel 24 by a predetermined decremental amount ΔPc2, for example, by about −1 to −20 mmHg. Then, the control cycle starting with the above-described S2 and followed by the subsequent steps is repeatedly executed, so that the diameter D and wall thickness T of the arterial vessel 44 are repeatedly calculated while the pressure Pc in the pressure vessel 24 is reduced from time to time. This control cycle corresponds to a period from the point of time “b” to a point of time “d” through a point of time “c” indicated in FIGS. 5 and 6.

When the pressure Pc in the pressure vessel 24 has been reduced to the lower limit of −80 mmHg (when the trans-wall pressure Pa has been raised to the maximum value (Ps+80 mmHg) during repeated execution of the above-described control cycle, an affirmative determination is obtained in S11, so that the re-pressurization progress flag F1 is reset to 0, while the re-evacuation progress flag F2 is set to 1. This control cycle corresponds to a period from the point of time “d” to the point of time “a” indicated in FIGS. 5 and 6, Accordingly, an affirmative determination is obtained in S6 in the next execution of the control cycle starting with S2, and the control flow goes to S14 to determine whether the pressure Pc in the pressure vessel 24 has been raised to the initial value of 0 mmHg (atmospheric pressure). Since a negative determination is obtained in S14 when this step is implemented for the first time, the control flow goes to S15 corresponding to the above-described pressure control portion 74, to raise the pressure Pc in the pressure vessel 24 by the predetermined incremental amount ΔPc1, for example, by about 1-20 mmHg. Then, the control cycle starting with the above-described S2 and followed by the subsequent steps is repeatedly executed, so that the diameter D and wall thickness T of the arterial vessel 44 are repeatedly calculated while the pressure Pc in the pressure vessel 24 is raised from time to time. This control cycle corresponds to a period from the point of time “d” to the point of time “a” indicated in FIGS. 5 and 6.

When the pressure Pc in the pressure vessel 24 has been raised to the initial value of 0 mmHg during repeated execution of the above-described control cycle, an affirmative determination is obtained in S14, so that the control flow goes to S16 corresponding to the evaluation value calculating portion 82, to calculate the stiffness parameter β, press-strain elasticity coefficient Ep, arterial-vessel-diameter change rate AS, compliance value DC, compliance value CC, incremental elasticity coefficient E_{inc}, blood vessel shrinkage ratio SR, time constant τ upon shrinkage of the blood vessel, and the ratios ΔK and/or ratio ΔS. Then, S17 corresponding to the display control portion 80 is implemented to display, on the display 16, the evaluation values calculated in S16, as indicated in FIG. 6, and also the graph of FIG. 6 indicative of a change of the blood vessel diameter D with the trans-wall pressure P_{A}, the graph of FIG. 7 indicative of a change of the blood vessel wall thickness T with the trans-wall pressure P_{A}, the graph of FIG. 8 indicative of a change of the blood vessel diameter D with the pressure Pc in the pressure vessel 24, and the graph of FIG. 9 indicative of a change of the blood vessel wall thickness T with the pressure Pc, all together, or selectively according to a manual selecting operation, on the basis of the data stored in the memory portion 72.

The vital luminal part evaluating apparatus 10 according to the present embodiment described above is arranged such that the pressure vessel 24 is provided with the annular inflation bag 24f (first sealing device) and the annular inflation bag 24g (second sealing device) for sealing the pressure vessel 24 at an intermediate position of the brachium 34 (first position) and an intermediate position of the antebrachium 22 (second position) in the longitudinal direction of the limb (arms) of the live body, and is configured to permit a change of an internal pressure therein over a pressure range a lower limit of which is a negative value, while a portion of the brachium and antebrachium between first and second positions in the longitudinal direction of the arms is accommodated in the pressure vessel 24, so that the pressure vessel 24 can be comparatively small-sized even where arterial vessel 44 (luminal part) of a comparatively large diameter is accommodated in the pressure vessel 24, whereby the physical and mental burden on the subject person can be reduced. The reduction of the physical and metal burden permits stable measurement of a cross sectional shape of the arterial vessel 44 (luminal part), and consequently permits accurate evaluation of the vital luminal part. It is particularly noted that since the subject person can see the distal part of the limb passed through the pressure vessel 24, the subject person can be given a high degree of metal stability.

The vital luminal part evaluating apparatus 10 according to the present embodiment is further arranged such that the pair of annular inflation bags 24f, 24g are provided as the first sealing device and the second sealing device, and these annular inflation bags 24f, 24g are inflated for sealing at the first position and/or the second position of the arms of the live body, so that the pressure vessel 24 can be sealed with respect to the external space with high stability; irrespective of a dimensional variation of the subject portion of the live body due to sexual, age and physical differences of the live body.

The vital luminal part evaluating apparatus 10 according to the present embodiment is further arranged such that the diameter (cross sectional shape value) D of the arterial vessel 44 in the brachium 34 accommodated in the pressure vessel 24 is measured by a non-invasion method by the blood-vessel-diameter calculating portion (cross sectional shape measuring device) 76 in the process of a change of the internal pressure in the pressure vessel 24 over the pressure range the lower limit of which is a negative value, while the portion of the subject person 20 between the antebrachium 22 and the brachium 34 is accommodated in the pressure vessel 24, and a change of the internal pressure Pc in the pressure vessel 24 and a change of the diameter D of the arterial vessel 44 which takes place with the change of the internal pressure Pc in the pressure vessel 24 are displayed on the display 16 under the control of the display control portion (display control means) 80. Since the pressure in the pressure vessel 24 accommodating the brachium 34 is thus changed over the pressure range the lower limit of which is the negative value, the upper limit of the trans-wall pressure P_{A} of the arterial vessel 44 which is conventionally limited to the value corresponding to the systolic blood pressure is raised to a value of about 200 mmHg sufficiently higher than the systolic blood pressure, so that the diameter D of the arterial vessel 44 obtained in a high-pressure region of the trans-wall pressure P_{A} can be used to display on the display 16 the change of the internal pressure Pc in the pressure vessel 24, and the change of the diameter D of the arterial vessel 44 with the change of the internal pressure Pc in the pressure vessel 24, namely, the dynamic characteristics of the arterial vessel 44, and to accurately evaluate the arterial vessel 44 on the basis of the dynamic characteristics. That is, the elastic characteristics of the arterial vessel 44 can be detected in the high-pressure region of the trans-wall pressure P_{A} not lower than the systolic blood pressure, so that the elastic characteristics can be accurately obtained, permitting a sufficiently high degree of accuracy of diagnosis in terms of the arteriosclerosis. The upper limit of the trans-wall pressure P_{A} of the arterial vessel 44 which is raised to provide the high-pressure region makes it possible to implement the measurement and evaluation while the diameter of the arterial vessel 44 is enlarged, leading to a further improvement of the measurement accuracy and evaluation accuracy.

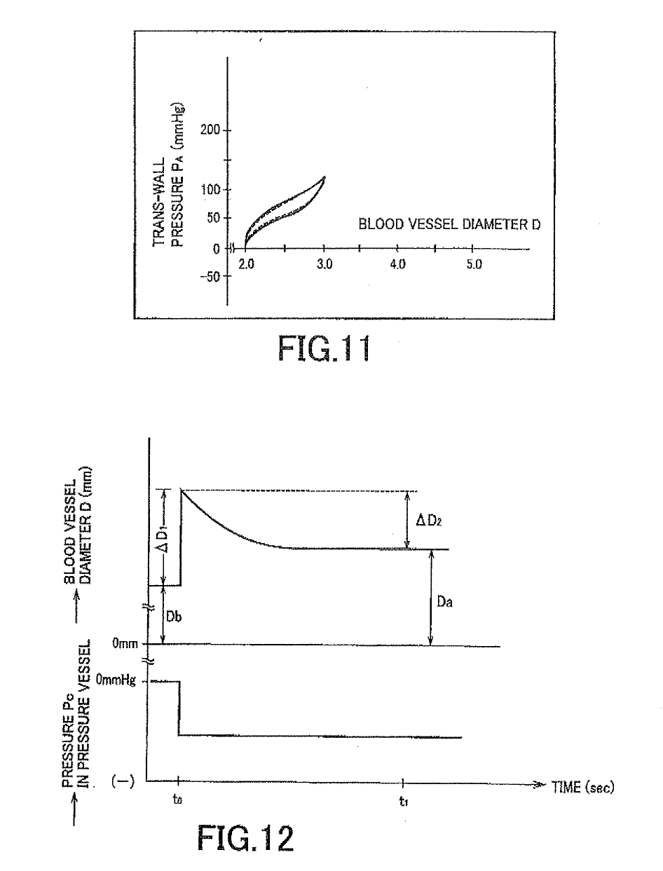

In connection with the above, reference is made to FIG. 11 indicating a change of the diameter D of the arterial vessel 44 with a change of the depression pressure acting on the arterial vessel 44, as indicated in FIG. 6, during measurement of elastic characteristics of the blood vessel wall by a conventional apparatus, on the basis of the change of the arterial vessel diameter with a change of a pressure acting on the blood vessel wall (trans-wall pressure), which is a difference between the blood pressure and the depression pressure with which a subject portion of a live body is depressed by water-inflated bags. According to this conventional apparatus, the upper limit of the trans-wall pressure P_{A} does not exceed the systolic blood pressure Ps, so that the measurement is not possible in a high-pressure region around 200 mmHg, whereby the elastic characteristics of an atherosclerotic patient indicated by solid lines and the elastic characteristics of a normal person indicated by broken lines cannot be distinguished from each other. Thus, the conventional apparatus does not permit the measurement and evaluation with a sufficiently high degree of accuracy. The blood vessel diameter D indicated in FIG. 11 is normalized by a radius of the blood vessel at 0 mmHg, as in FIG. 6.

The vital luminal part evaluating apparatus 10 according to the present embodiment is further arranged such that the display control portion (display control means) 80 commands the display 16 to continuously display a plurality of points indicative of a change of the internal pressure Pc in the pressure vessel 24 and a change of the diameter (cross sectional shape) D of the arterial vessel 44 with the change of the internal pressure Pc in the pressure vessel 24, in a two-dimensional coordinate system in which the diameter (cross sectional shape) D of the arterial vessel 44 is taken along an axis while the internal pressure Pc in the pressure vessel 24 is taken along another axis. Accordingly, the dynamic characteristics of the arterial vessel 44 can be obtained on the basis of the points displayed on the display 16, and the arterial vessel 44 can be accurately evaluated on the basis of the obtained dynamic characteristics.

The vital luminal part evaluating apparatus 10 according to the present embodiment is further arranged such that the display control portion (display control means) 80 commands the display 16 to display the internal pressure Pc in the pressure vessel 24 and the diameter (cross sectional shape) D of the arterial vessel 44 continuously along the axis of time, making it possible to obtain the internal pressure Pc in the pressure vessel 24 and the diameter D of the arterial vessel 44 during the measurement, for easy determination of an abnormality of the measurement or rapid treatment of the abnormality.

The vital luminal part evaluating apparatus 10 according to the present embodiment is further arranged so as to include the display control portion (display control means) 80 configured to change the internal pressure Pc in the pressure vessel 24, between a predetermined negative minimum pressure value (e.g., −80 mmHg) and a positive maximum pressure value (e.g., 200 mmHg) predetermined to be not lower than the systolic blood pressure Ps of the subject person 20, so that the high-pressure region of the range of the trans-wall pressure P_{A} of the arterial vessel 44 can be set as desired by changing the minimum pressure value, to measure the dynamic characteristics of the arterial vessel 44 in the high-pressure region.

The vital luminal part evaluating apparatus 10 according to the present embodiment is further arranged such that the blood-vessel-diameter calculating portion (cross sectional shape measuring device) 76 measures the diameter D and wall thickness T of the arterial vessel 44 on the basis of the reflected ultrasonic wave signal SR received from the brachium 34 of the subject person 20, so that the dynamic characteristics of the arterial vessel 44 can be accurately obtained on the basis of the measured values.

The vital luminal part evaluating apparatus 10 according to the present embodiment is further arranged such that the diameter D and wall thickness T of the arterial vessel 44 in the antebrachium 22 of the subject person 20 accommodated in the pressure vessel 24 are measured by the non-invasion method by the blood-vessel-diameter calculating portion 76 and blood-vessel-wail-thickness calculating portion 78 (cross sectional shape measuring device) in the process of a change of the internal pressure Pc in the pressure vessel 24 over the pressure range the lower limit of which is a negative value, while the brachium 34 of the subject person 20 is accommodated in the pressure vessel 24, and the evaluation values indicative of the dynamic characteristics of the arterial vessel 44 are calculated by the evaluation value calculating portion (evaluation value calculating means) 82 on the basis of a change of the diameter D of the arterial vessel 44 which takes place with a change of the internal pressure Pc in the pressure vessel 24, so that the evaluation values indicative of the dynamic characteristics of the arterial vessel 44 calculated by the evaluation value calculating portion 82 are displayed under the control of the display control portion (output means) 80. Since the pressure in the pressure vessel 24 accommodating the brachium 34 of the subject person 20 is thus changed over the pressure range the lower limit of which is the negative value, the upper limit of the trans-wall pressure PA of the arterial vessel 44 which is conventionally limited to the value corresponding to the systolic blood pressure is raised to a value of about 200 mmHg sufficiently higher than the systolic blood pressure, so that the cross sectional shape of the arterial vessel 44 obtained in a high-pressure region of the trans-wall pressure P_{A} can be used to calculate the evaluation values indicative of the dynamic characteristics of the arterial vessel 44 on the basis of the change of the internal pressure Pc in the pressure vessel 24, and the change of the diameter D of the arterial vessel 44 with the change of the internal pressure Pc in the pressure vessel 24, whereby the arterial vessel 44 can be accurately evaluated on the basis of the dynamic characteristics. That is, the elastic characteristics of the arterial vessel 44 can be detected in the high-pressure region of the trans-wall pressure P_{A} not lower than the systolic blood pressure, so that the elastic characteristics can be accurately obtained, permitting a sufficiently high degree of accuracy of diagnosis in terms of the arteriosclerosis. The upper limit of the trans-wall pressure of the arterial vessel 44 which is raised to provide the high-pressure region makes it possible to implement the measurement and evaluation while the diameter D of the arterial vessel 44 is enlarged, leading to a further improvement of the measurement accuracy and evaluation accuracy.

The vital luminal part evaluating apparatus 10 according to the present embodiment is further arranged such that the evaluation value calculating portion 82 calculates, as an evaluation value or values indicative of the dynamic characteristics of the arterial vessel 44, at least one of the stiffness parameter β, press-strain elasticity coefficient Ep, arterial-vessel-diameter change rate AS, compliance value DC, compliance value CC, incremental elasticity coefficient E_{inc}, blood vessel shrinkage ratio SR, and time constant upon shrinkage of the blood vessel, on the basis of a change of the diameter D of the arterial vessel 44 which takes place with a change of the internal pressure Pc in the pressure vessel 24, so that the dynamic characteristics of the arterial vessel 44 can be accurately obtained on the basis of the calculated value or values.

The vital luminal part evaluating apparatus 10 according to the present embodiment is further arranged such that the evaluation value calculating portion 82 calculates, as evaluation values indicative of the dynamic characteristics of the arterial vessel 44, the differences or ratios ΔK of the evaluation values (stiffness parameter β, press-strain elasticity coefficient Ep, arterial-vessel-diameter change rate AS, compliance value DC, compliance value CC, incremental elasticity coefficient E_{inc}, blood vessel shrinkage ratio SR and time constant τ upon shrinkage of the blood vessel) indicative of the dynamic characteristics of the arterial vessel 44 obtained in the predetermined high-pressure region of the trans-wall pressure P_{A} not lower than 120-150 mmHg, for example, with respect to those obtained in a predetermined low-pressure region of the trans-wall pressure P_{A} not higher than 80 mmHg, for example, so that the arterial vessel 44 can be accurately evaluated in terms of arteriosclerosis on the basis of the differences or ratios ΔK.

The vital luminal part evaluating apparatus 10 according to the present embodiment is further arranged such that the evaluation value calculating portion 82 calculates, as an evaluation value indicative of the dynamic characteristics of the arterial vessel 44, the ratio ΔS of the amount of increase ΔD⁺ of the diameter D of the arterial vessel 44 when the pressure in the pressure vessel 24 is reduced by a predetermined amount, with respect to the amount of decrease ΔD⁻ of the diameter D of the arterial vessel when the pressure in the pressure vessel 24 is raised by a predetermined amount, so that the arterial vessel 44 can be accurately evaluated in terms of arteriosclerosis o the basis of the calculated ratio ΔS.

The vital luminal part evaluating apparatus 10 according to the present embodiment is further arranged such that the blood-vessel-diameter calculating portion (cross sectional shape measuring device) 76 measures the diameter D of the arterial vessel 44 on the basis of the reflected ultrasonic wave signal SR received from the antebrachium 22 of the subject person 20, so that the dynamic characteristics of the arterial vessel 44 can be accurately obtained by changing the internal pressure in the pressure vessel according to the measured diameter D of the arterial vessel 44.

### Embodiment 2

Another embodiment of this invention will be described next. In the following description, same reference signs are used for the same elements in the different embodiments, which will not be described.

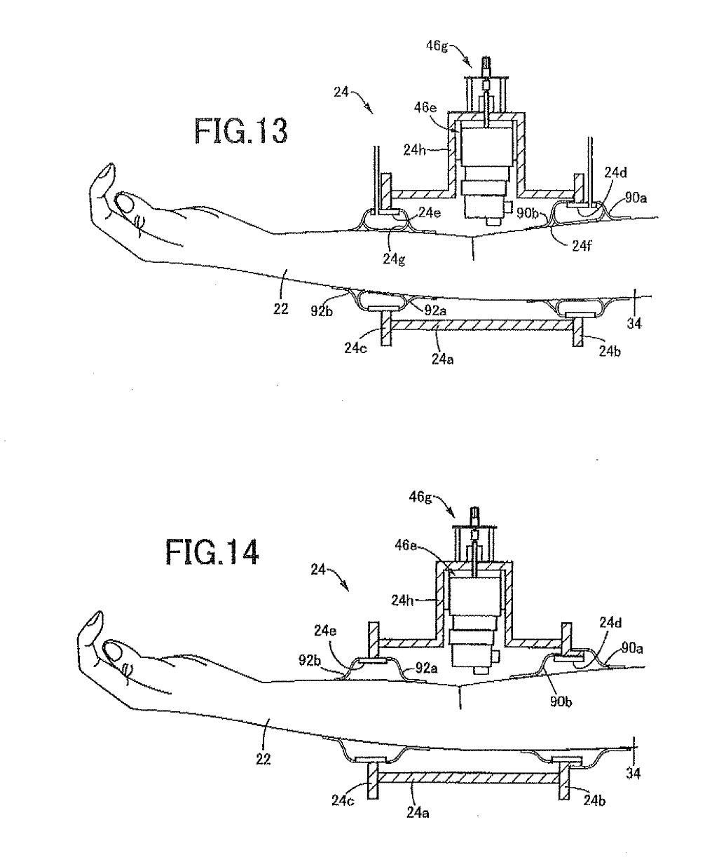

FIG. 13 is the longitudinal cross sectional view showing a modified arrangement of sealing devices provided in the pressure vessel 24. In the embodiment of FIG. 13, too, the pressure vessel 24 is provided with the pair of annular inflation bags 24f, 24g formed of a soft resin or synthetic rubber material and fixed to the inner circumferential surfaces of the through-holes 24d, 24e, for sealing between the through-hole 24d and the brachium 34 and between the through-hole 24e and the antebrachium 22. However, the pressure vessel 24 are further provided with two pairs of flexible annular films 90a, 90b, and 92a, 92b, which are disposed inside and outside of the pressure vessel 24, that is, the annular inflation bags 24f, 24g and which have radially inner end portions having width dimensions sufficient for surface contact with the brachium 34 and antebrachium 22. Each of these pairs of flexible annular films 90a, 90b, 92a, 92b is formed from a rubber sheet of a comparatively small thickness, for example. In the present embodiment wherein the pair of flexible annular films 90a, 90b, 92a, 92b are disposed on the opposite sides of each of the annular inflation bag 24f, 24g, the pressure vessel 24 is sealed with respect to the brachium 34 and antebrachium 22 of the live body, based on a pressure difference between the pressure within the pressure vessel 24 and the atmospheric pressure, so that the stability of sealing between the inside and outside of the pressure vessel 24 is further improved irrespective of a dimensional variation of the subject portion of the live body due to sexual, age and physical differences of the live body.

### Embodiment 3

FIG. 14 is the longitudinal cross sectional view showing another modified arrangement of sealing devices provided in the pressure vessel 24. Unlike the embodiment of FIG. 13, the present embodiment of FIG. 14 does not have the pair of annular inflation bags 24f, 24g, and is configured to establish sealing between the pressure vessel 24 and the brachium 34 and antebrachium 22, with the two pairs of flexible annular films 90a, 90b, 92a, 92b. In the present embodiment, the sealing devices are simplified in construction, and do not require the pump 24k and pressure control valve 24m.

While the embodiments of this invention have been described by reference to the drawings, it is to be understood that the invention may be otherwise embodied.

In the embodiments of FIGS. 2, 13 and 14, for example, the sealing device for sealing between the through-hole 24d of the pressure vessel 24 and the brachium 34 is identical in construction with that for sealing between the through-hole 24e and the antebrachium 22. However, these two sealing devices have different constructions. For instance, the sealing device of FIG. 13 or 14 is used for sealing between the through-hole 24d of the pressure vessel 24 and the brachium 34, while the sealing device of FIG. 2 is used for sealing between the through-hole 24e of the pressure vessel 24 and the antebrachium 22.

In the embodiments of FIGS. 13 and 14, the pair of flexible annular films 90a, 90b is provided for sealing between the through-hole 24d of the pressure vessel 24 and the brachium 34, while the pair of flexible annular films 92a, 92b is provided for sealing between the through-hole 24e and the antebrachium 22. However, only one of the flexible annular films 90a, 90b, and only one of the flexible annular films 92a, 92b may be used. For instance, only the flexible annular film 90a is provided for sealing between the pressure vessel 24 and the brachium 34, while only the flexible annular film 92b is provided for sealing between the pressure vessel 24 and the antebrachium 22.

While the pressure vessel 24 in the embodiments of FIGS. 2, 13 and 14 is configured for sealing with respect to the antebrachium 22 and brachium 34, the pressure vessel may be configured for sealing with respect to a portion of one of the lower limbs.

Although the blood pressure measuring portion 68 in the embodiments described above uses the cuff 36 for the blood pressure measurement, the pressure vessel 24 may be used, in place of the cuff 36, for depressing the brachium 34 for the blood pressure measurement by the oscillometric method.

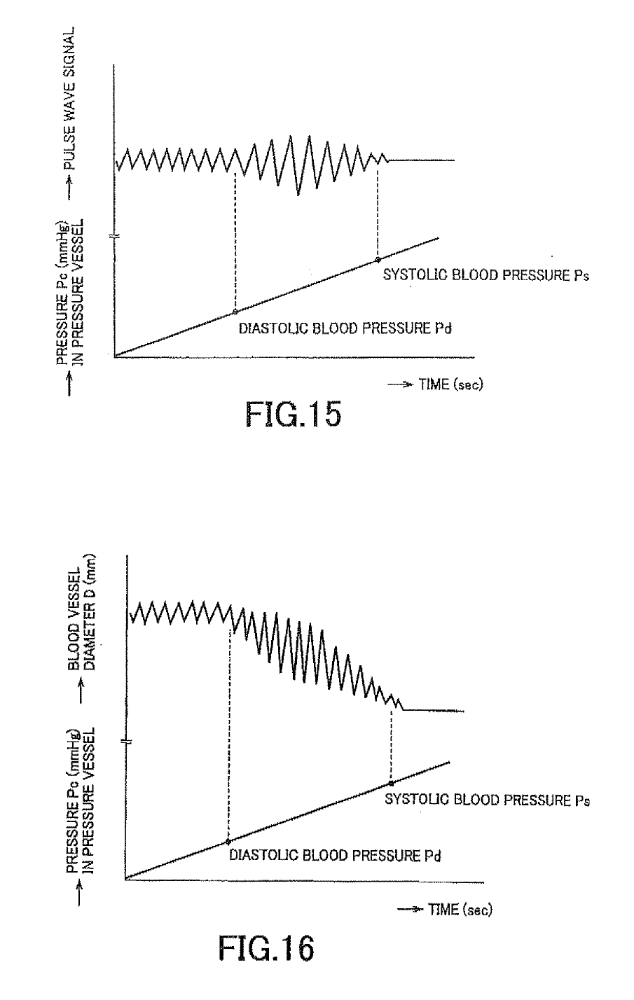

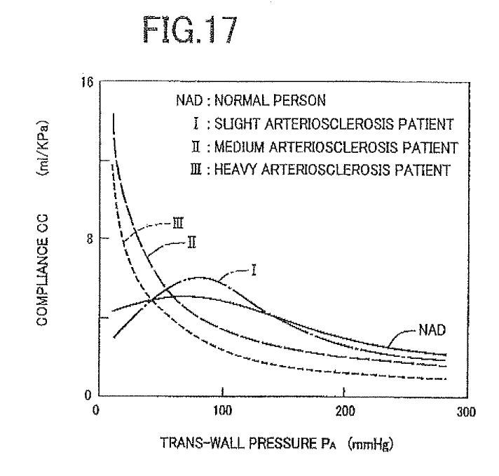

The pressure measuring portion 68 in the embodiments described above is configured to gradually raise the pressure Pc in the pressure vessel 24 at a predetermined rate to a value higher than the systolic blood pressure by a predetermined amount, and determines in the process of rise of the pressure Pc, as the diastolic and systolic blood pressures, the values of the pressure Pc in the pressure vessel 24 at which a difference (a rate of change) of the amplitude of the pressure pulsation or pulse wave included in the pressure Pc is maximum, as indicated in FIG. 15. However, the pressure measuring portion 68 may be configured to determine in the process of gradual rise of the pressure Pc in the pressure vessel 24 at the predetermined rate to the value higher than the systolic blood pressure by the predetermined amount, as the diastolic and systolic blood pressures, the values of the pressure Pc at which a difference of the amplitude of a wave indicative of the diameter D of the arterial vessel 44 is maximum, as indicated in FIG. 16. This modification has not only the advantages as described above with respect to the vital luminal part evaluating apparatus according to the illustrated embodiments, but also an advantage that a % FMD can be measured on the basis of a rate of change of the blood vessel diameter D, as well as an advantage that the cuff 36, pressure control valve 40 and other devices exclusively used for the blood pressure measurement in the illustrated embodiments can be eliminated.

While the present invention has been described for illustrative purpose only, it is to be understood that the invention may be embodied with various changes and improvements, which may occur to those skilled in the art.

### NOMENCLATURE OF REFERENCE SIGNS

10: Vital luminal part evaluating apparatus
20: Subject person (Live body)
22: Antebrachium (Limb)
24: Pressure vessel
24f, 24g: Annular inflation bags (First and second sealing devices)
34: Brachium (Limb)
44: Arterial vessel (Luminal part)
46: Ultrasonic wave probe (Cross sectional shape measuring device)
76: Blood-vessel-diameter calculating portion (Cross sectional shape measuring device)
90a, 92b: Flexible annular film (First and second sealing devices)

1. A vital luminal part evaluating apparatus provided with a pressure vessel configured to permit a change of an internal pressure within a pressure range a lower limit of which is a negative value therein while the pressure vessel accommodates a portion of a live body, and a luminal cross sectional shape measuring device configured to measure, by a non-invasion method, a cross sectional shape value of a luminal part in the portion of the live body accommodated in said pressure vessel, for evaluating said luminal part located in said portion of the live body, on the basis of the cross sectional shape value of the luminal part, characterized in that:
said pressure vessel is provided with a first sealing device and a second sealing device for sealing the pressure vessel at respective first and second positions in a longitudinal direction of a limb of said live body, and is configured to permit the change of the internal pressure over the pressure range, while a portion of the limb of said live body between the first and second positions is accommodated in the pressure vessel.

2. The vital luminal part evaluating apparatus according to claim 1, wherein at least one of said first sealing device and said second sealing device is provided with an annular inflation bag, which is inflated for sealing the pressure vessel at corresponding one of said first and second positions of the limb of said live body

3. The vital luminal part evaluating apparatus according to claim 1, wherein at least one of said first sealing device and said second sealing device is provided with a pair of flexible annular films which are disposed inside and outside of said pressure vessel and which have radially inner end portions having width dimensions sufficient for surface contact with said limb, for sealing the pressure vessel with respect to the limb of said live body at a corresponding of said first and second positions of the limb, based on a pressure difference between the internal pressure within said pressure vessel and an atmospheric pressure.
